(19) United States
(12) Patent Application Publication
(10) Pub. No.: US 2023/0069062 A1
(43) Pub. Date: Mar. 2, 2023

(54) X-RAY DIAGNOSIS APPARATUS, MEDICAL INFORMATION PROCESSING APPARATUS AND METHOD, AND STORAGE MEDIUM

(71) Applicant: CANON KABUSHIKI KAISHA, Tokyo (JP)

(72) Inventors: Tomoki Fujito, Tochigi (JP); Haruki Iwai, JP (JP); Takeshi Noda, Kanagawa (JP); Sota Torii, Tokyo (JP); Katsuro Takenaka, Saitama (JP)

(21) Appl. No.: 17/822,093

(22) Filed: Aug. 24, 2022

(30) Foreign Application Priority Data
Aug. 31, 2021 (JP) 2021-141010

(51) Int. Cl. A61B 6/00

(52) U.S. Cl. CPC A61B 6/463; A61B 6/482; A61B 6/505; A61B 6/5282; A61B 6/5294; A61B 6/4241

(57) ABSTRACT

An X-ray diagnosis apparatus controls to display information based on an error in an index value evaluating a state of a bone of an object based on at least one of a captured image of the object and an imaging condition which correspond to X-rays with two different types of energies.

### BACKGROUND OF THE INVENTION

### Field of the Invention

The embodiments disclosed in this specification and the accompanying drawings relate to an X-ray diagnosis apparatus, a medical information processing apparatus and method, and a storage medium.

### Description of the Related ART

Conventionally, as a technique for measuring an index indicating the state of the bone, such as a bone mineral density (BMD) or bone mineral content (BMC), a dual-energy X-ray absorptiometry (DXA) method is known. The DXA method discriminates the bone and the soft tissue by using captured image data of an object corresponding to X-rays with two different types of energies and calculating the bone mineral density and the bone mineral content by the discrimination.

As an apparatus that measures an index indicating the state of the bone, a dedicated X-ray diagnosis apparatus (to be referred to as a dedicated apparatus hereinafter) is known. The dedicated apparatus reduces the influence of scattered rays by imaging narrow stripe-shaped X-ray irradiation regions while sequentially moving the X-ray irradiation regions in their short side direction. Recently, X-ray diagnosis apparatuses (to be referred to as general-purpose apparatuses hereinafter) other than dedicated apparatuses also have performed bone mineral density/bone mineral content measurement using the DXA method.

Although the above dedicated apparatuses and general-purpose apparatuses display bone mineral densities and bone mineral contents, such display does not allow proper evaluation of time-series changes in bone mineral density/bone mineral content.

### CITATION LIST

### Patent Literature

Japanese Patent Laid-Open No. 2018-192054

### Non Patent Literature

“Proximal femur BMD measurement manual”, [Searched on Mar. 1, 2021], Internet <http://www.josteo.com/ja/guideline/doc/4_1.pdf>

One of the problems that the embodiments disclosed in this specification and the accompanying drawings intend to solve is how to properly evaluate a time-series change in the state of the bone of an object. However, the problems that the embodiments disclosed in this specification and the accompanying drawings intend to solve are not limited to the above problem. The problems corresponding to the effects of the respective arrangements disclosed in the embodiments described below can be regarded as other problems.

### SUMMARY OF THE INVENTION

According to one aspect of the present invention, there is provided an X-ray diagnosis apparatus comprising a display control unit configured to display information based on an error in an index value evaluating a state of a bone of an object based on at least one of a captured image of the object and an imaging condition which correspond to X-rays with two different types of energies.

According to another aspect of the present invention, there is provided a medical information processing apparatus comprising a display control unit configured to display information based on an error in an index value evaluating a state of a bone of an object based on at least one of a captured image of the object and an imaging condition which correspond to X-rays with two different types of energies.

According to another aspect of the present invention, there is provided a medical information processing method comprising displaying information based on an error in an index value evaluating a state of a bone of an object based on at least one of a captured image of the object and an imaging condition which correspond to X-rays with two different types of energies.

Further features of the present invention will become apparent from the following description of exemplary embodiments with reference to the attached drawings.

### BRIEF DESCRIPTION OF THE DRAWINGS

FIG. 1 is a block diagram showing an example of the arrangement of an X-ray diagnosis apparatus according to the first embodiment;

FIG. 2 is a flowchart showing an outline of a processing procedure by the X-ray diagnosis apparatus according to the first embodiment;

FIG. 3 is a flowchart showing a processing procedure by a calculation function and a control function according to the first embodiment;

FIG. 4 is a view for explaining an example of processing by the calculation function according to the first embodiment;

FIG. 5 is a view showing an example of information displayed by the control function according to the first embodiment;

FIG. 6 is a view showing an example of information displayed by the control function according to the first embodiment;

FIG. 7 is a flowchart showing a processing procedure by a calculation function and a control function according to the second embodiment;

FIG. 8 is a view showing an example of information displayed by the control function according to the second embodiment;

FIG. 9 is a view showing an example of information displayed by the control function according to the second embodiment;

FIG. 10 is a block diagram showing an example of the arrangement of an X-ray diagnosis apparatus according to the third embodiment;

FIG. 11 is a view for explaining an example of processing by a calculation function and a control function according to other embodiments; and

FIG. 12 is a block diagram showing an example of the arrangement of a medical information processing apparatus according to other embodiments.

### DESCRIPTION OF THE EMBODIMENTS

The embodiments of X-ray diagnosis apparatuses and medical information processing apparatuses will be described in detail below with reference to the accompanying drawings. X-ray diagnosis apparatuses and medical information processing apparatuses according to the present application are not limited by the embodiments described below. In addition, in the following description, common reference numerals denote the same constituent elements, and redundant descriptions will be omitted.

### First Embodiment

The arrangement of an X-ray diagnosis apparatus according to the first embodiment will be described. The X-ray diagnosis apparatus according to this embodiment measures an index evaluating the state of the bone by the DXA method. Indices evaluating the state of the bone include a bone mineral density (BMD) and a bone mineral content (BMC). X-ray diagnosis apparatuses include dedicated apparatuses that measure the state of the bone and general-purpose apparatuses such as X-ray TV apparatuses and X-ray general-purpose imaging apparatuses. Note that the first embodiment exemplifies a C-arm type X-ray TV apparatus as a general-purpose apparatus.

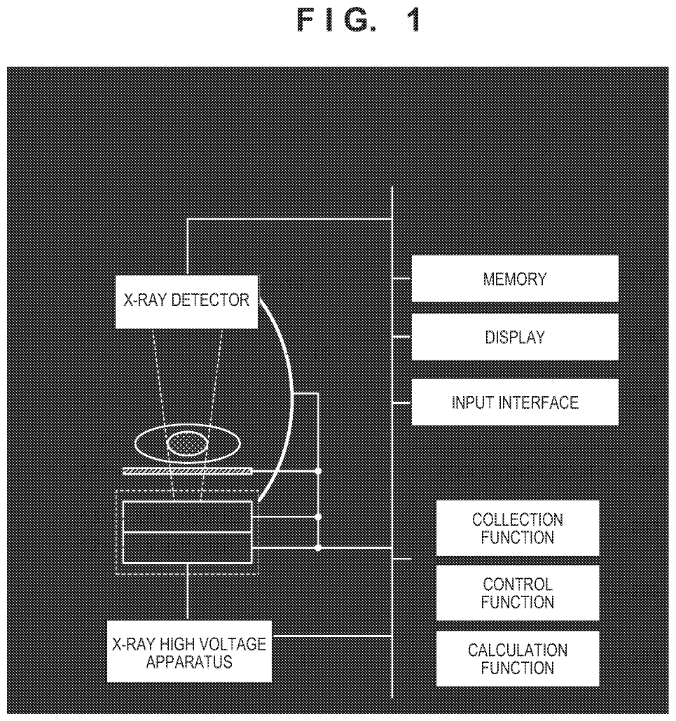

FIG. 1 is a block diagram showing an example of the arrangement of an X-ray diagnosis apparatus 1 according to the first embodiment. As shown in FIG. 1, the X-ray diagnosis apparatus 1 includes an X-ray high voltage apparatus 11, an X-ray tube 12, an X-ray aperture 13, a top 14, a C-arm 15, an X-ray detector 16, a memory 17, a display 18, an input interface 19, and a processing circuit 20.

The X-ray high voltage apparatus 11 applies a high voltage to the X-ray tube 12 under the control of the processing circuit 20. For example, the X-ray high voltage apparatus 11 includes a transformer, electric circuits such as a rectifier, a high voltage generator that generates a high voltage applied to the X-ray tube 12, and an X-ray controller that controls an output voltage in accordance with the X-rays applied by the X-ray tube 12. Note that the high voltage generator may be of a transformer type or inverter type.

The X-ray tube 12 is a vacuum tube including a cathode (filament) that generates thermions and an anode (target) that generates X-rays upon impingement of thermions. The X-ray tube 12 generates X-rays by applying thermions from the cathode to the anode using a high voltage applied from the X-ray high voltage apparatus 11.

The X-ray aperture 13 includes an X-ray aperture element that narrows the irradiation range of X-rays generated by the X-ray tube 12 and a filter that adjusts the X-rays emitted from the X-ray tube 12.

The X-ray aperture element of the X-ray aperture 13 includes, for example, four slidable aperture blades. Sliding the aperture blades makes the X-ray aperture irradiate an object P with the X-rays generated by the X-ray tube 12 upon narrowing the X-rays. In this case, the aperture blades are plate-like members made of lead or the like and are provided near the X-ray irradiation port of the X-ray tube 12 to adjust the X-ray irradiation range. In addition, the aperture blades may be formed such that the opposing blades are asymmetrically movable. Alternatively, the aperture blades may be formed such that the opposing blades are only symmetrically movable.

In order to reduce the exposure dose of the object P and improve the image quality of X-ray image data, the filter of the X-ray aperture 13 changes the radiation quality of transmitted X-rays by changing the material and its thickness, reduces soft radiation components that are easily absorbed by the object P, and reduces high-energy components that cause a deterioration in the contrast of X-ray image data. In addition, the filter changes the dose and irradiation range of X-rays by changing the material, thickness, position, and the like and attenuates X-rays so as to make the X-rays applied from the X-ray tube 12 onto the object P have a predetermined distribution.

For example, the X-ray aperture 13 has a drive mechanism such as a motor or actuator and controls irradiation with X-rays by operating the drive mechanism under the control of the processing circuit 20. For example, the X-ray aperture 13 adjusts the opening degree of the aperture blades of the X-ray aperture element and controls the irradiation range of X-rays applied onto the object P by applying a drive voltage to the drive mechanism in accordance with a control signal received from the processing circuit 20. For example, the X-ray aperture 13 adjusts the position of the filter to control the distribution of X-rays applied onto the object P by applying a drive voltage to the drive mechanism in accordance with a control signal received from the processing circuit 20.

The top 14 is a bed on which the object P is placed and is arranged on a gantry (not shown). For example, the gantry has a drive mechanism such as a motor or actuator and moves and tilts the top 14 by moving the drive mechanism under the control of the processing circuit 20.

The C-arm 15 holds the X-ray tube 12, the X-ray aperture 13, and the X-ray detector 16 so as to make them face each other through the object P. For example, the C-arm 15 has a drive mechanism such as a motor or actuator and controls the irradiation position and irradiation angle of X-rays by rotating and moving the X-ray tube 12, the X-ray aperture 13, and the X-ray detector 16 relative to the object P by applying a drive voltage to the drive mechanism in accordance with a control signal received from the processing circuit 20.

The X-ray detector 16 is, for example, an X-ray flat panel detector (FPD) having detection elements arrayed in a matrix pattern. The X-ray detector 16 detects X-rays emitted from the X-ray tube 12 and transmitted through the object P and outputs a detection signal corresponding to the amount of X-rays detected to the processing circuit 20. Note that the X-ray detector 16 may be an indirect conversion type detector including a grid, a scintillator array, and a photosensor array or a direct conversion type detector including semiconductor elements that convert incident X-rays into electrical signals.

The memory 17 is implemented by, for example, a semiconductor memory device such as a random access memory (RAM). The memory 17 temporarily stores the processing result obtained by the processing circuit 20. For example, the memory 17 receives and temporarily stores various data such as X-ray image data collected by the processing circuit 20. In this case, X-ray image data according to the present application includes the detection signal detected by the X-ray detector 16, the projection data generated based on the detection signal, and the X-ray image generated based on the projection data. In addition, the memory 17 stores programs corresponding to the respective functions which are read out and executed by the processing circuit 20.

The display 18 display various information. For example, the display 18 displays a GUI for receiving an instruction from the operator and various X-ray images under the control of the processing circuit 20. The display 18 also displays the processing result obtained by the processing circuit 20. For example, the display 18 displays the measured value of an index (bone mineral density, bone mineral content, or the like) for evaluating the state of the bone of the object P, an error in the measured value, and the like.

The input interface 19 receives various input operations from the operator, converts the received input operations into electrical signals, and outputs them to the processing circuit 20. For example, the input interface 19 is implemented by a mouse and a keyboard, a trackball, switches, buttons, a joystick, a touch pad that performs input operations based on touches on the operation screen, a touch screen as a combination of a display screen and a touch pad, a non-contact input circuit using an optical sensor, a speech input circuit, and the like. Note that the input interface 19 may be formed from a tablet terminal or the like that can wirelessly communicate with the apparatus main body. The input interface 19 is not limited to the one including physical operation components such as a mouse and a keyboard. Examples of the input interface 19 include an electrical signal processing circuit that receives an electrical signal corresponding to an input operation from an external input device provided separately from the apparatus and outputs the electrical signal to the processing circuit 20.

The processing circuit 20 controls the overall operation of the X-ray diagnosis apparatus 1. In addition, the processing circuit 20 functions as a collection function 201, a control function 202, and a calculation function 203 by reading out and executing programs stored in the memory 17. The control function 202 is an example of a display control unit. The calculation function 203 is an example of a calculation unit.

Here, it is important to properly evaluate a time-series change in the state of the bone of an object. However, when a measured value in a past examination is compared with a measured value in the current examination, displaying measured index values alone does not allow, at a first glance, the understanding of whether the time-series change is a significant difference. This makes it impossible to properly evaluate a time-series change in the state of the bone of the object.

The X-ray diagnosis apparatus 1 having the above arrangement displays both an index value evaluating the state of the bone of the object and an error in the index value. This makes it possible to properly evaluate a time-series change in the state of the bone of the object.

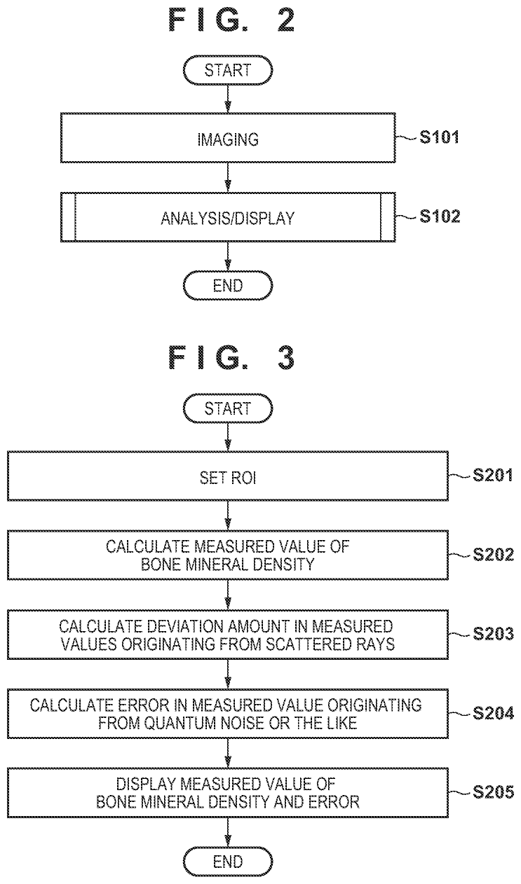

The processing executed by the X-ray diagnosis apparatus 1 will be described below with reference to FIG. 2. FIG. 2 is a flowchart showing an outline of a processing procedure by the X-ray diagnosis apparatus 1.

The collection function 201 decides X-ray conditions for imaging by dual energy. For example, the collection function 201 decides X-ray conditions input from the input interface 19 or X-ray conditions (for example, set at a previous examination) stored in the memory 17 as X-ray conditions for imaging. When the operator issues an instruction to execute imaging via the input interface 19, the collection function 201 executes imaging by dual energy in accordance with the decided X-ray conditions.

Note that the collection function 201 can also decide X-ray conditions for imaging by dual energy based on fluoroscopic positioning. In such a case, first of all, the collection function 201 sequentially executes the collection of fluoroscopic images to perform positioning in accordance with the execution of a fluoroscopic operation by the operator. In this case, the collection function 201 executes automatic brightness control (ABC) with respect to collected fluoroscopic images, for example, comparing an average pixel value with a threshold and feedbacking the comparison result to X-ray conditions for the next fluoroscopic image.

When the X-ray conditions are stabilized by ABC control, the collection function 201 makes the memory 17 hold body thickness information based on the X-ray conditions as “information concerning the body thickness” of the object. The collection function 201 also decides X-ray conditions for imaging by dual energy (imaging with two different types of tube voltages) based on the “information concerning the body thickness”. Note that the collection function 201 may decide X-ray conditions for imaging by dual energy based on X-ray conditions that have reached a stable condition by ABC control. When the operator issues an instruction to execute imaging, the collection function 201 executes imaging in accordance with the decided X-ray conditions (step S101).

For example, the collection function 201 performs imaging with a first tube voltage (high voltage) which is targeted at a region including a region of interest (ROI) such as the lumbar spine or the proximal femur and collects X-ray image data corresponding to the first tube voltage. In addition, the collection function 201 performs imaging with a second tube voltage (low voltage) which is targeted at the same region including the ROI and collects X-ray image data corresponding to the second tube voltage.

Note that imaging for collecting captured images of an object which correspond to X-rays with two different types of energies is not limited to imaging by dual energy described above. For example, this imaging may be imaging with one irradiation with X-rays of continuous X-ray energy by using a two-layer detector that detects low-energy X-rays and high-energy X-rays by dispersing the continuous X-ray energy.

When the collection function 201 executes imaging, the calculation function 203 calculates an index value evaluating the state of the bone of the object from two types of X-ray image data collected by imaging by dual energy. The control function 202 displays the calculated index value (for example, a bone mineral density or bone mineral content) (step S102). For example, the calculation function 203 generates a bone image based on the two types of X-ray image data and measures a bone mineral density, bone mineral content, or the like in a region of interest of the generated bone image. The control function 202 makes the display 18 display the measured bone mineral density, bone mineral content, or the like.

In step S102, the calculation function 203 calculates an error in the calculated index value (bone mineral density, bone mineral content, or the like). The control function 202 displays the calculated error in the index value (bone mineral density, offset correction, or the like).

An example of processing in step S102 will be described below with reference to FIG. 3. The following exemplifies a bone mineral density (BMD) as an index evaluating the state of the bone of an object.

The calculation function 203 generates a bone image from the two types of X-ray image data collected by imaging by dual energy. In addition, the calculation function 203 sets an ROI with respect to the generated bone image (step S201).

The calculation function 203 may set the ROI designated by the operator. Alternatively, the calculation function 203 may set the ROI extracted from a bone image as a result of existing segmentation processing. Note that X-ray image data for which an ROI is set by the calculation function 203 may be the high-kV image collected by imaging with the first tube voltage (high voltage), the low-kV image collected by imaging with the second tube voltage (low voltage), or a bone enhanced image (to be described later). Using the high-kV image or bone enhanced image indicating a bone region more clearly makes it possible to set a more accurate ROI. When a bone enhanced image is to be used, substance discrimination processing (to be described later) is executed before ROI setting.

Subsequently, the calculation function 203 measures a bone mineral density based on the projection data (high-kV image) collected by imaging with the first tube voltage (high voltage) and the projection data (low-kV image) collected by imaging with the second tube voltage (low voltage) (step S202).

More specifically, the calculation function 203 obtains the distribution of linear attenuation coefficients concerning each of the two types of projection data and solves simultaneous equations based on the linear attenuation coefficients of two substances (the bone and a substance (soft tissue) other than the bone) and the mixture amount at each position (each pixel) in the distribution of the linear attenuation coefficients to calculate the mixture amount or mixture ratio of the two substances at each position. The calculation function 203 also generates two types of projection data respectively corresponding to the bone and the soft tissue based on the mixture amount of the bone and the soft tissue at each position. Note that the memory 17 stores the projection data generated by the collection function 201.

The calculation function 203 also solves the simultaneous equations based on the linear attenuation coefficients and the densities of the two substances (the bone and the substance (soft tissue) other than the bone) at each position (each pixel) to calculate the densities of the two substances at each position. For example, the calculation function 203 calculates a bone mineral density (BMDm) in an ROI.

The calculation function 203 calculates an error in an index value (bone mineral density) based on at least the captured images of the object or imaging conditions which correspond to X-rays with the two different types of energies. In this case, the calculation function 203 calculates an error in the measured value of the bone mineral density obtained by single DXA imaging or single imaging using a two-layer detector. Note that bone mineral density errors according to the first embodiment include an error originating from scattered rays included in a captured image and an error originating from quantum noise or the like.

An example of calculating an error in an index value (bone mineral density) based on a captured image and imaging conditions will be described below. Note that the imaging conditions include X-ray conditions (including a tube voltage, a tube current, and a pulse width) in single DXA imaging (or single imaging using a two-layer detector), geometric imaging conditions, and information concerning the body thickness of an object.

Error Originating from Scattered Rays

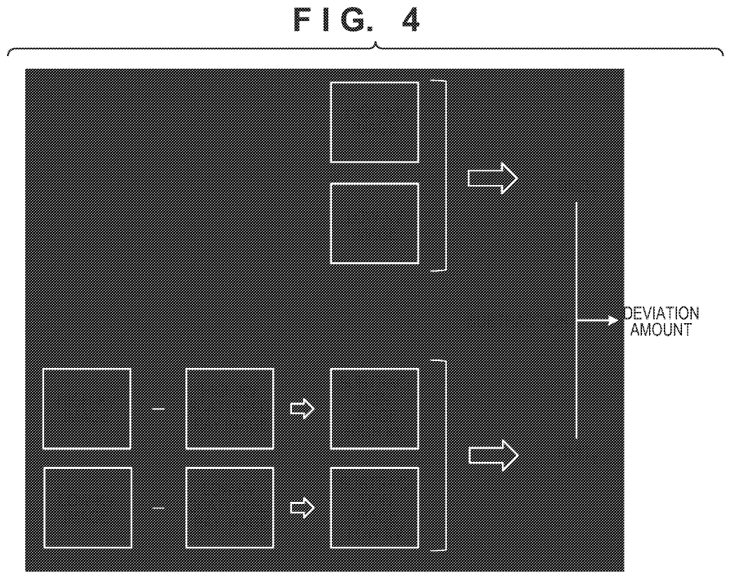

The calculation function 203 obtains an error originating from scattered rays included in a captured image (step S203). As shown in FIG. 4, an error originating from scattered rays included in a captured image is obtained from the difference between the bone mineral density measured based on an image (original image) with no reduction in scattered rays and the bone mineral density measured based on an image with a reduction in scattered rays.

More specifically, the calculation function 203 obtains a scattered ray image (high-kV scattered ray image) concerning X-ray image data (high-kV image) corresponding to the first tube voltage (high voltage) and a scattered ray image (low-kV scattered ray image) concerning X-ray image data (low-kV image) corresponding to the second tube voltage (low voltage). The amount of scattered rays (scattered ray image) at each pixel is obtained based on X-ray conditions (kV and mAs), geometric imaging conditions, and information concerning the body thickness of the object.

Geometric imaging conditions include the position information of the aperture blades, a source to image receptor distance (SID), and a source-to-object distance (SOD) (or a detector/top distance). The position information of the aperture blades is represented by four parameters when the aperture blades can move asymmetrically or represented by two parameters when the aperture blades can move only symmetrically. Geometric imaging conditions in imaging are recorded in the DICOM tag of a high-kV image and/or a low-kV image captured by imaging.

In addition, as information concerning the body thickness of an object, “information concerning the body thickness” obtained by ABC control may be used. Alternatively, this information may be calculated by using X-ray image data collected by dual energy imaging.

The memory 17 stores, in advance, for example, scattered ray data indicating the relationship between X-ray conditions, geometric imaging conditions and body thickness, and scattered rays. The calculation function 203 obtains “high-kV scattered ray image” and “low-kV scattered ray image” by estimating the amount of scattered rays based on scattered ray data with respect to each dual energy imaging. The calculation function 203 estimates “high-kV scattered ray image” shown in FIG. 4 based on the X-ray conditions, the geometric imaging conditions, and the information concerning the body thickness of the object in imaging with the high voltage. Likewise, the calculation function 203 estimates “low-kV scattered ray image” shown in FIG. 4 based on the X-ray conditions, the geometric imaging conditions, and the information concerning the body thickness of the object in imaging with the low voltage.

The calculation function 203 then generates each subtraction image (subtraction image high kV and subtraction image low kV) by excluding the high-kV scattered ray image from the high-kV image and excluding the low-kV scattered ray image from the low-kV image. The calculation function 203 sets an ROI based on the position and size of the ROI set in step 5201 with respect to the bone image (the image with a reduction in scattered ray) generated from each subtraction image and calculates a bone mineral density in the set ROI. The calculation function 203 obtains the difference between the bone mineral density in the ROI of the image with no reduction in scattered ray and the bone mineral density in the ROI of the image with a reduction in scattered ray as an error originating from the scattered rays included in the captured image.

Error Originating from Quantum Noise or Like

Referring back to FIG. 3, upon calculating the error originating from the scattered rays in the above manner, the calculation function 203 obtains an error in the measured value originating from quantum noise or the like (step S204). An error originating from quantum noise or the like is, for example, an error originating from quantum noise or circuit noise. An error originating from quantum noise is an error originating from variations in X-rays which probabilistically occur in the irradiation path of X-rays. An error originating from circuit noise is an error originating from variations in operation which probabilistically occur in a circuit included in the X-ray diagnosis apparatus 1.

The calculation function 203 calculates, as an error in a measured value, a value evaluating variations in value based on a captured image. For example, the calculation function 203 may calculate, with respect to a plurality of ROIs (for example, the vertebral body), statistic information such as the standard deviation or variance of the bone mineral density measured for each ROI as an error originating from quantum noise or the like.

The calculation function 203 may calculate the sum of a quantum noise amount and a circuit noise amount as an error originating from quantum noise or the like. For example, the calculation function 203 obtains the number of photons in an X-ray image (for example, a high-kV image or low-kV image collected by dual energy imaging) based on pixel values and a circuit noise amount of the X-ray image and estimates a quantum noise amount based on the number of photons. A circuit noise amount is calculated based on a signal obtained without irradiation with X-rays.

In addition, the calculation function 203 may calculate a quantum noise amount based on a bone enhanced image and a soft tissue enhanced image generated based on substance discrimination processing as an error originating from quantum noise or the like. For example, the calculation function 203 obtains the number of photons incident on each pixel by simulation using a bone enhanced image and a soft tissue enhanced image and estimates a quantum noise amount based on the number of photons.

In this manner, in accordance with the calculation of an error originating from quantum noise or the like, the control function 202 displays information based on the calculated error in the index value. For example, the control function 202 makes the display 18 display the measured value of a bone mineral density together with the error (step S205). Such errors include an error originating from scattered rays included in a captured image and an error originating from quantum noise or the like.

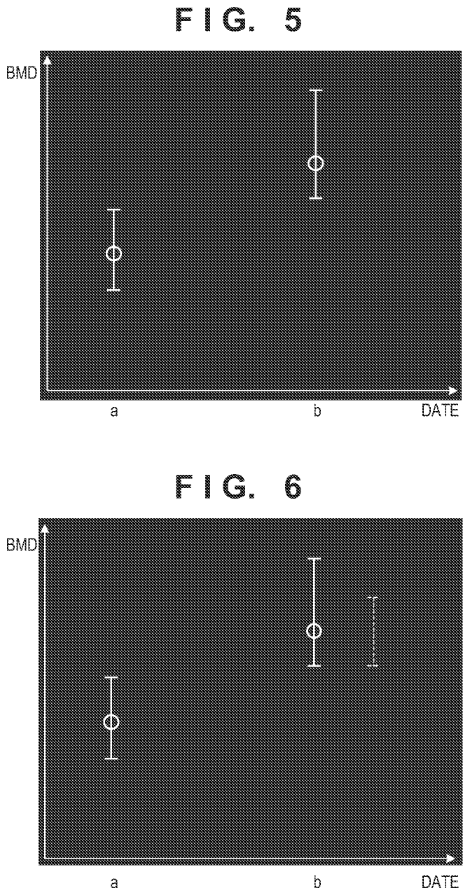

FIG. 5 is a view showing an example of information displayed on the display 18. The ordinate indicates bone mineral density (BMD), and the abscissa indicates examination date (Date).

For example, the control function 202 displays the measured values of bone mineral densities at “Date: a” and “Date: b” and error bars incorporating at least one of “error originating from scattered rays” and “error originating from quantum noise or the like”. The upper side of each error bar relative to the measured values includes “error originating from scattered rays” because it includes scattered rays and hence the bone mineral density is estimated low. In addition, the upper and lower sides of each error bar relative to the measured values include “error originating from quantum noise or the like”. Note that the information based on the error in the index value displayed by the control function 202 is not limited to the information indicating the value of the error described above and may be information derived based on the error. For example, the control function 202 may compare a calculated error and an error reference value and display the comparison result (for example, information indicating that the error exceeding the reference value).

Note that the control function 202 may display an error when a condition for the estimation of an error in a measured value is changed. An example of calculating an error in an index value (bone mineral density) based on imaging conditions will be described below. In such a case, the calculation function 203 obtains an error in a measured value when a geometric imaging condition is changed. More specifically, the calculation function 203 obtains an error in the measured value of a bone mineral density based on geometric imaging conditions in the imaging conditions. For example, the calculation function 203 estimates the amount of scattered rays entering an ROI based on the geometric imaging conditions and estimates an error in the measured value based on the estimated amount of scattered rays. For example, the calculation function 203 calculates “error originating from scattered rays” when a condition such as the position information of the aperture blades, SID, or SOD is changed. The control function 202 displays an error in a measured value when a geometric imaging condition is changed.

FIG. 6 is a view showing an example of information displayed on the display 18 when a geometric imaging condition is changed. The following exemplifies the case in which an error in a measured value is displayed when a geometric imaging condition is changed at “Date: b” in FIG. 5.

For example, the control function 202 displays an error in a measured value (the error bar indicated by the dotted line) when a geometric imaging condition is changed next to the display of the measured value at “Date: b” and the error. Note that the mode of displaying errors in measured values in a simulation in which a geometric imaging condition is changed is not limited to the example shown in FIG. 6, and various other modes can be used to display such errors. For example, the control function 202 can display error bars indicated in different colors and shapes identifiable from errors based on actual conditions so as to superimpose the error bars on measured values. Alternatively, the control function 202 can perform display on another window together with a GUI for changing a geometric imaging condition.

The above embodiment has exemplified the case in which a bone mineral density is used as an index value evaluating the state of the bone of an object. However, the embodiment is not limited to this and may use a bone mineral content as an index value evaluating the state of the bone of an object.

As described above, according to the first embodiment, the calculation function 203 calculates an error in an index value evaluating the state of the bone of an object based on at least captured images of the object or imaging conditions which correspond to X-rays with two different types of energies. The control function 202 displays information based on the calculated error in the index value. Accordingly, the X-ray diagnosis apparatus 1 according to the first embodiment can display an error in a measured value for each examination in bone mineral density/bone mineral content examination processing and allows proper evaluation of a time-series change in the state of the bone of the object.

According to the first embodiment, the calculation function 203 calculates an error in an index value evaluating the state of the bone of an object based on geometric imaging conditions in the imaging conditions. Therefore, when making a general-purpose apparatus capable of variously changing geometric imaging conditions calculate measured values of a bone mineral density and a bone mineral content, the X-ray diagnosis apparatus 1 can accurately estimate errors in the measured values.

When, for example, the general-purpose apparatus performs a bone mineral density/bone mineral content examination, the amount of scattered rays entering an ROI changes depending on the geometric imaging conditions (the opening degree of the aperture blades, SID, SOD, and the like). The error amounts in the measured values change with the change in the amount of scattered rays. The X-ray diagnosis apparatus 1 according to the first embodiment can estimate an error based on geometric imaging conditions and hence can accurately calculate an error in measurement by such a general-purpose apparatus.

According to the first embodiment, the calculation function 203 calculates statistic information in a captured image as an error in an index value evaluating the state of the bone of the object. Accordingly, the X-ray diagnosis apparatus 1 according to the first embodiment can calculate an error in a measured value based on a captured image.

According to the first embodiment, the calculation function 203 estimates the amount of scattered rays entering an ROI based on geometric imaging conditions and estimates an error in an index value based on the estimated amount of scattered rays. Therefore, the X-ray diagnosis apparatus 1 according to the first embodiment can present an error in an index value in consideration of an error originating from scattered rays.

According to the first embodiment, the calculation function 203 estimates an error including an error based on quantum noise. Accordingly, the X-ray diagnosis apparatus 1 according to the first embodiment can present an error in an index value in consideration of an error originating from quantum noise.

According to the first embodiment, the calculation function 203 calculates an error in an index value when a geometric imaging condition is changed. The control function 202 displays an error in an index value when a geometric imaging condition is changed. Accordingly, the X-ray diagnosis apparatus 1 according to the first embodiment can execute various simulations concerning geometric imaging conditions with respect to errors in index values.

### (First Modification)

The above embodiment has exemplified the case in which an index value (bone mineral density) calculated based on captured images before scattered ray correction (the high-kV image and the low-kV image in FIG. 4) is set as a measured value, and the difference between the measured value and an index value (bone mineral density) calculated based on captured images after scattered ray correction (the subtraction image high kV and the subtraction image low kV in FIG. 4) is obtained as an error in the measured value. However, the embodiment is not limited to this. For example, an index value (bone mineral density, bone mineral content, or the like) calculated based on captured images after scattered ray correction (the subtraction image high kV and the subtraction image low kV in FIG. 4) may be set as a measured value, and the difference between the measured value and an index value calculated based on captured images before scattered ray correction may be obtained as an error in the measured value. In such a case, an error originating from scattered rays is included in the lower side of the error bar relative to the measured value. Note that when an index value calculated based on captured images after scattered ray correction is displayed, only an error originating from quantum noise or the like may be displayed without including an error originating from scattered rays.

### (Second Modification)

The above embodiment has exemplified the case in which the amount of scattered rays is estimated based on scattered ray data indicating the relationship between X-ray conditions, geometric imaging conditions and the body thickness, and scattered rays, and “high-kV scattered ray image” and “low-kV scattered ray image” are obtained. However, the embodiment is not limited to this, and “high-kV scattered ray image” and “low-kV scattered ray image” may be obtained by artificial intelligence (AI). In such a case, for example, a learned model is generated in advance by using a captured image and a scattered ray image based on the captured image as learning data and is stored in the memory 17. The calculation function 203 obtains “high-kV scattered ray image” and “low-kV scattered ray image” by inputting captured images (a high-kV image and a low-kV image) to the learned model. This makes it possible to display an error in an index value by using only a captured image without using any imaging conditions.

### Second Embodiment

The first embodiment described above has exemplified the case in which errors in index values include an error originating from scattered rays and an error originating from quantum noise or the like. The second embodiment exemplifies a case in which errors in index values include an estimation accuracy error in the amount of scattered rays and an error originating from quantum noise or the like. An X-ray diagnosis apparatus 1 according to the second embodiment differs from the X-ray diagnosis apparatus 1 according to the first embodiment in the contents of processing by a calculation function 203. The following description will focus on this difference.

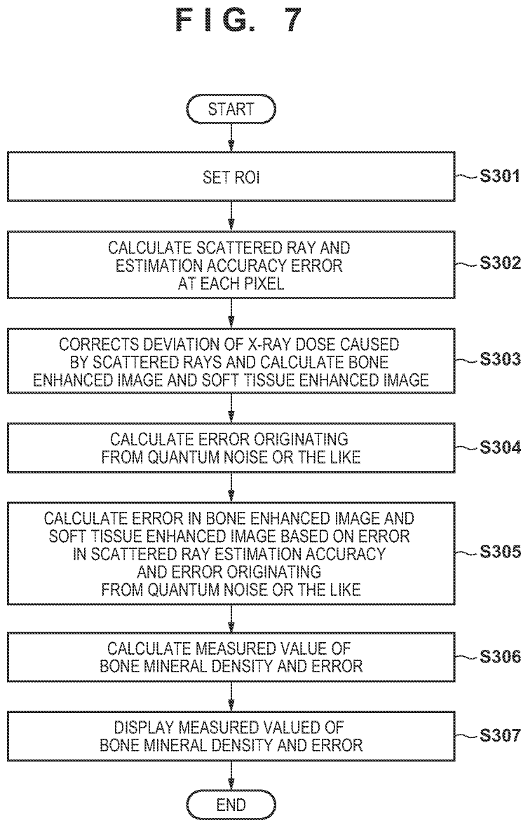

A processing procedure according to the second embodiment will be described below with reference to FIG. 7. Note that FIG. 7 shows the details of the processing in step S102 in FIG. 2. The following will exemplify a bone mineral density (BMD) as an index evaluating the state of the bone of the object.

For example, first of all, as shown in FIG. 7, an X-ray diagnosis apparatus 1 according to the second embodiment makes the calculation function 203 set an ROI with respect to a collected X-ray image as in the first embodiment (step S301).

Subsequently, the calculation function 203 calculates the amount of scattered rays and an error in the scattered ray estimation accuracy at each pixel with respect to two types of X-ray image data collected by dual energy imaging (step S302).

### (Error in Scattered Ray Estimation Accuracy)

The calculation function 203 calculates an error in scattered ray estimation accuracy based on imaging conditions. More specifically, the calculation function 203 calculates “error in scattered ray estimation accuracy” originating from the deviation between a value used for the estimation of the amount of scattered rays and an actual value in information concerning the X-ray conditions and the body thickness. For example, estimation accuracy data indicating a range that can be estimated as the amount of scattered rays for each condition is generated in advance and stored in the memory 17. The calculation function 203 calculates a range that can be regarded as the amount of scattered rays based on the estimation accuracy data for each dual energy imaging. The calculation function 203 according to the second embodiment calculates an error in a measured value based on the range that can be regarded as the amount of scattered rays described above.

For example, the calculation function 203 estimates an error in a measured value by statistic processing using a range that can be estimated as the amount of scattered rays calculated based on the imaging conditions for “high-kV image” and a range that can be estimated as the amount of scattered rays calculated based on the imaging conditions for “low-kV image”. For example, the calculation function 203 estimates, as an error in a measured value, the maximum or minimum range of ranges based on the imaging conditions for “high-kV image” and “low-kV image”.

Subsequently, the calculation function 203 corrects the deviation of the X-ray dose caused by scattered rays and calculates a bone enhanced image and a soft tissue enhanced image (step S303). More specifically, the calculation function 203 generates subtraction images (a subtraction image high kV and a subtraction image low kV) by subtracting corresponding scattered ray images from a high-kV image and a low-kV image. A bone enhanced image and a soft tissue enhanced image are generated based on substance discrimination processing using each subtraction image. Note that processing for scattered ray images respectively corresponding to the high-kV image and the low-kV image is executed by using one of the techniques described in the first embodiment.

The calculation function 203 then calculates “error originating from quantum noise or the like” (step S304). Note that this step is executed by using one of the techniques described in the first embodiment as in step S204 in FIG. 3.

Subsequently, the calculation function 203 calculates a bone enhanced image and a soft tissue enhanced image based on “error in scattered ray estimation accuracy” and “error originating from quantum noise or the like” (step S305). More specifically, the calculation function 203 calculates errors in the bone enhanced image and the soft tissue enhanced image by using a numerical technique or analytic technique using “error in scattered ray estimation accuracy” and “error originating from quantum noise or the like”.

The calculation function 203 then calculates a measured value of the bone mineral density and its error (step S306). More specifically, the calculation function 203 calculates the measured value of the bone mineral density based on the substance discrimination processing executed in step S303. The calculation function 203 also calculates an error in the measured value of the bone mineral density based on the error in the enhanced image calculated in step S305.

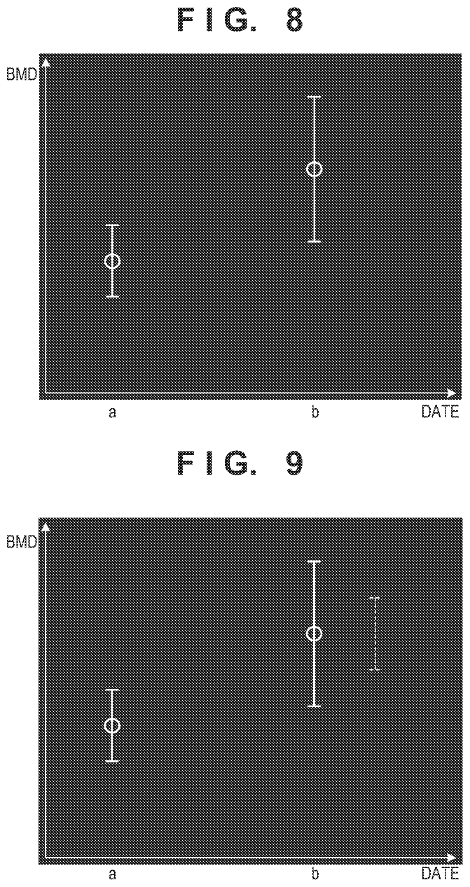

As described above, when the measured value of a bone mineral density and an error are calculated, a control function 202 makes a display 18 display the measured value of the bone mineral density and the error (step S307). FIG. 8 is a view showing an example of information displayed on the display 18. The ordinate indicates bone mineral density (BMD), and the abscissa indicates examination date (Date).

For example, the control function 202 displays the measured values of bone mineral densities at “Date: a” and “Date: b” and error bars indicating errors in the measured values of the bone mineral densities calculated based on “estimation accuracy error in the amount of scattered rays” and “error originating from quantum noise or the like”.

Note that the control function 202 can display an error in a measured value when a geometric imaging condition is changed as in the first embodiment. That is, the calculation function 203 calculates “estimation accuracy error in the amount of scattered rays” when conditions such as the position information of the aperture blades, SID, and SOD are changed. The calculation function 203 then calculates an error in the measured value of the bone mineral density by using the calculated “estimation accuracy error in the amount of scattered rays”.

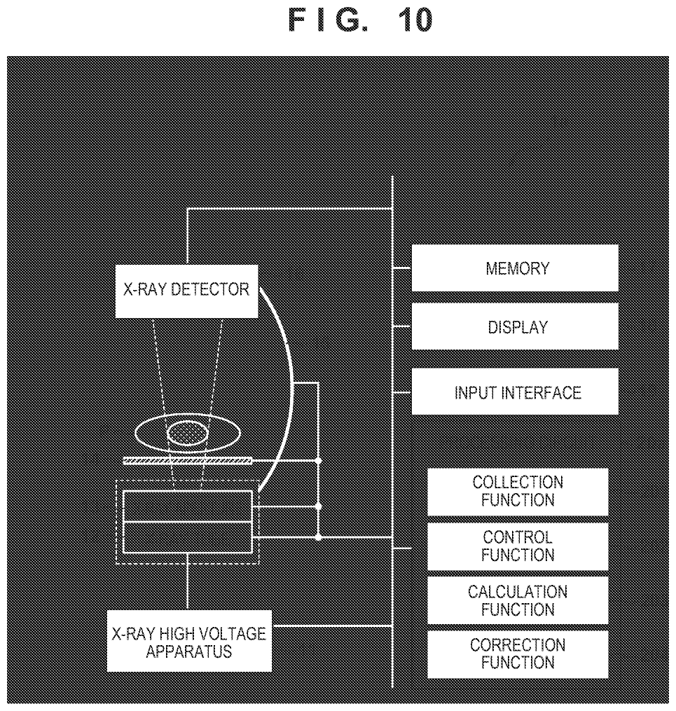

The control function 202 displays an error in a measured value when a geometric imaging condition is changed. FIG. 9 is a view showing an example of information displayed on the display 18 when a geometric imaging condition is changed and shows an example of displaying an error in a measured value when a geometric imaging condition at “Date: b” in FIG. 8 is changed. For example, the control function 202 displays an error in a measured value (the error bar indicated by the dotted line) when the measured value at “Date: b” and the error are displayed side by side and a geometric imaging condition is changed. Note that in the second embodiment as in the first embodiment, errors in measured values in a simulation in which a geometric imaging condition is changed can be displayed in various modes.

The above embodiment has exemplified the case in which a bone mineral density is used as an index value evaluating the state of the bone of an object. However, the embodiment is not limited to this, and a bone mineral content may be used as an index value evaluating the state of the bone of an object.

As described above, according to the second embodiment, the calculation function 203 estimates an error in an index value by using an estimation accuracy error in the amount of scattered rays. Therefore, the X-ray diagnosis apparatus 1 according to the second embodiment can calculate an error corresponding to an estimation error in the amount of scattered rays.

### Third Embodiment

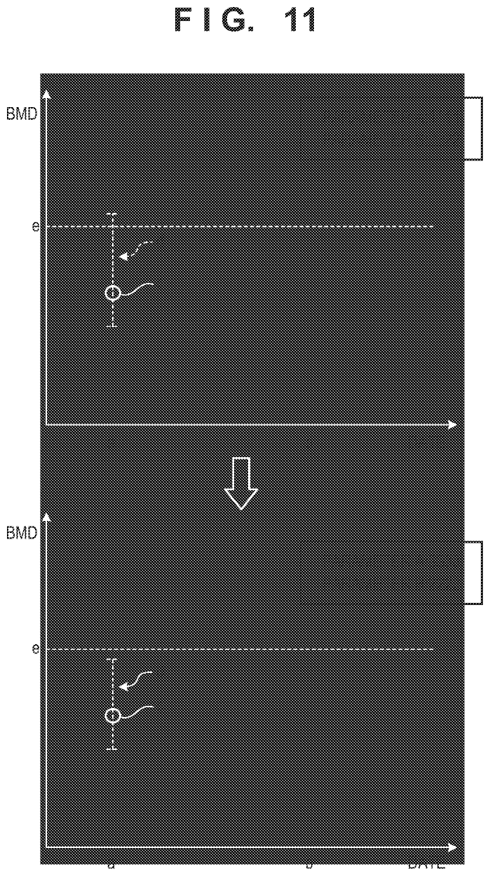

The third embodiment exemplifies a case in which scattered rays are estimated by using pixel values in a region shielded from X-rays by the aperture blades. That is, the third embodiment calculates an error originating from scattered rays based on an image. FIG. 10 is a block diagram showing an example of the arrangement of an X-ray diagnosis apparatus la according to the third embodiment. The X-ray diagnosis apparatus la according to the third embodiment differs from the X-ray diagnosis apparatus 1 according to the first embodiment in that a processing circuit 20a newly executes a correction function 204 and in the contents of processing by a calculation function 203. The following description will focus on these differences. Note that the correction function 204 is an example of the correction unit.

The correction function 204 corrects the amount of scattered rays in an ROI based on pixel values in a region other than an X-ray irradiation region determined by the X-ray aperture element. More specifically, first of all, the correction function 204 calculates the amount of scattered rays in a region shielded from X-rays by the aperture blades while setting, as a signal originating from scattered rays, a detection signal detected in the region shielded from X-rays.

The correction function 204 then generates a coordinate-dependent scattered ray function in an X-ray irradiation region (a region that is not shielded from X-rays by the aperture blades) based on the scattered ray image estimated by the calculation function 203 and corrects the generated scattered ray function by using the amount of scattered rays in the region shielded from X-rays. For example, the correction function 204 multiplies the scattered ray function by a constant such that the amount of scattered rays based on the coordinate-dependent scattered ray function described above is continuous on the boundary of X-ray irradiation by the aperture blades (the boundary between a position where X-rays are applied and a position where X-rays are blocked).

The correction function 204 calculates the amount of scattered rays at each position in an X-ray irradiation region based on the corrected scattered ray function. The calculation function 203 calculates an error in the measured value by using the amount of scattered rays calculated by the correction function 204. Note that the scattered ray image estimated by the calculation function 203 may be the one estimated based on imaging conditions or may be estimated by using AI.

As described above, the X-ray diagnosis apparatus la according to the third embodiment can estimate scattered rays by using pixel values in a region shielded from X-rays by the aperture blades. In addition, the X-ray diagnosis apparatus la according to the third embodiment may estimate the amount of scattered rays based on the position of an ROI.

More specifically, the calculation function 203 estimates scattered rays in accordance with the opening degree of the aperture blades or the distance from an ROI to each aperture blade. When, for example, the opening degree of the aperture blades or the distance from an ROI to each aperture blade is larger than a threshold, the calculation function 203 estimates the amount of scattered rays by a technique described in the first embodiment. In this case, the calculation function 203 may use only the corrected the amount of scattered rays calculated by the correction function 204 described above.

In contrast, when the opening degree of the aperture blades or the distance from an ROI to each aperture blade is smaller than a threshold, the calculation function 203 calculates the amount of scattered rays in the ROI based on pixel values in a region other than the X-ray irradiation region determined by the X-ray aperture element. That is, the calculation function 203 estimates the amount of scattered rays in the ROI based on the detection signal detected in the region shielded from X-rays by the aperture blades. For example, the calculation function 203 calculates the amount of scattered rays in the region shielded from X-rays based on the detection signal detected in the region and uses the calculated amount of scattered rays as the amount of scattered rays in the ROI.

As described above, according to the third embodiment, the correction function 204 corrects the amount of scattered rays in the ROI based on pixel values in the region other than the X-ray irradiation region determined by the X-ray a perture element. Accordingly, the X-ray diagnosis apparatus la according to the third embodiment can correct the estimated amount of scattered rays based on an actually detected amount of scattered rays and hence can present more accurate errors.

According to the third embodiment, the calculation function 203 estimates the amount of scattered rays in the ROI based on the position of the ROI. Accordingly, the X-ray diagnosis apparatus la according to the third embodiment can estimate the amount of scattered rays corresponding to the position of the ROI.

According to the third embodiment, the calculation function 203 calculates the amount of scattered rays in the ROI based on pixel values in a region other than the X-ray irradiation region determined by the X-ray aperture element. Accordingly, the X-ray diagnosis apparatus la according to the third embodiment can estimate the amount of scattered rays using the actually detected amount of scattered rays.

### Other Embodiments

The first to third embodiments have been described so far, but the present invention can be executed in various different embodiments other than the first to third embodiments.

The above embodiments have exemplified the case in which the measured values of index values (bone mineral density, bone mineral content, and the like) and errors are displayed after dual energy imaging. However, the embodiments are not limited to this and may be applied to a case in which errors based on imaging conditions are calculated before imaging to determine whether the imaging conditions are proper, and the determination result is informed.

In such a case, a calculation function 203 calculates an error in an index value (bone mineral density, bone mineral content, or the like) based on imaging conditions used for single DXA imaging (or imaging conditions used for imaging with a two-layer detector) and executes determination on the imaging conditions based on the calculated error and the past measured value of the index value (bone mineral density, bone mineral content, or the like) corresponding to the error. A control function 202 displays the determination result.

In this case, the calculation function 203 can execute, as the above determination, determination on imaging conditions by comparison with an absolute value and determination on conditions concerning the evaluation of a change from a past index value (bone mineral density, bone mineral content, or the like). These operations will be described below.

When determining an imaging condition by comparison with an absolute value, the calculation function 203 calculates a typical error amount in the measured value of an index value (bone mineral density, bone mineral content, or the like) based on geometric imaging conditions and X-ray conditions. For example, the calculation function 203 obtains information concerning the past body thickness of the object as an examination target and calculates an error in the measured value of the index value (bone mineral density, bone mineral content, or the like) based on the obtained information concerning the past body thickness and the geometric imaging condition and the X-ray condition in the current imaging. Note that as the information concerning the body thickness of the object, the information estimated based on fluoroscopic positioning may be used. In addition, calculation of a typical error amount based on an imaging condition may be executed based on data with which a typical error amount is associated for each imaging condition and which is stored in the memory 17 in advance.

When adding the calculated typical error amount to the measured value of the past index value (bone mineral density, bone mineral content, or the like), the calculation function 203 determines whether there is any problem in terms of comparison with the absolute value. In this case, the absolute value is a young adult mean (YAM) value indicating the degree of reduction in bone mineral density when the bone mineral density of the young adult is 100%, the average value of bone mineral densities in an age group corresponding to the age of the object, an osteoporosis diagnosis threshold, or the like. That is, when comparing such an absolute value with a measured value, the calculation function 203 determines whether an error causes a problem in the comparing operation.

In addition, the calculation function 203 can recalculate a typical error amount in accordance with a change in imaging condition and determine whether a problem in terms of comparison with the absolute value is solved. Note that the typical error amount described above may be set to a large value in consideration of the amount of change from the past measured value of the index value (bone mineral density, bone mineral content, or the like) currently measured.

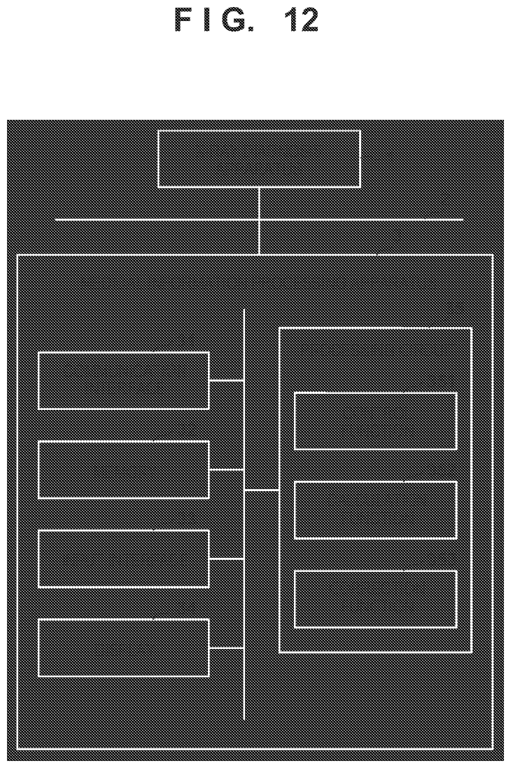

FIG. 11 is a view for explaining an example of processing by the calculation function 203 and the control function 202 according to other embodiments. The ordinate indicates bone mineral density (BMD), and the abscissa indicates examination date (Date). FIG. 11 shows a case in which determination on an imaging condition is executed before the examination at “Date: b”.

For example, the calculation function 203 calculates a typical error amount based on imaging conditions “parameter A: 1111 and parameter B: 2222” used for an examination at “Date: b”. The calculation function 203 then determines whether a threshold “e” is included in an error range when the typical error amount is added to the measured value of BMD in an examination at “Date: a”. That is, the calculation function 203 determines whether an error changes determination of whether the error range exceeds the threshold “e”.

For example, as shown in the upper graph in FIG. 11, when the threshold “e” is included in the error range, the calculation function 203 determines that there is a problem in terms of comparison with the threshold. The control function 202 makes the display 18 display, for example, the upper graph in FIG. 11 as a determination result. In this case, if the calculation function 203 determines that there is a problem in terms of comparison with the threshold, the control function 202 can make the display 18 further display information (for example, an alert) indicating that the imaging condition is not proper, in addition to the display of the upper graph in FIG. 11.

As shown in the lower graph in FIG. 11, the calculation function 203 then recalculates a typical error amount in accordance with the processing of changing “parameter A: 1111” of the imaging conditions to “parameter A: 3333”. The calculation function 203 further determines whether the threshold “e” is included in the error range, upon adding the typical error amount to the measured value of the BCD in the examination at “Date: a”. In this case, as shown in the lower graph in FIG. 11, if the threshold “e” is not included in the error range, the calculation function 203 determines that the imaging condition is proper. The control function 202 makes the display 18 display the lower graph in FIG. 11 in accordance with a change in imaging condition.

Note that an imaging condition may be changed based on an input operation by the operator or may be automatically changed by the calculation function 203 based on preset information.

When executing determination on a condition concerning the evaluation of a change from the measured value of a past index value (bone mineral density, bone mineral content, or the like), the calculation function 203 decides a threshold concerning an estimated error based on the measured value of the past index value (bone mineral density, bone mineral content, or the like) and the purpose of measurement. That is, the calculation function 203 decides a threshold (for example, the threshold e in FIG. 11) for determining whether an imaging condition is proper, based on the past measured value, the purpose, and the estimated error.

For example, the calculation function 203 sets a smaller threshold with a decrease in the measured value of an index value (bone mineral density, bone mineral content, or the like). In addition, the calculation function 203 sets a larger threshold when the purpose of measurement is a medical examination, sets a smaller threshold when the purpose is drug efficacy determination, and sets an intermediate threshold when the purpose is follow-up including no drug efficacy determination.

Note that the calculation function 203 may execute both or one of the determination on an imaging condition by comparison with an absolute value and the determination on a condition concerning the evaluation of a change from the measured value of a past index value (bone mineral density, bone mineral content, or the like).

The above embodiments have exemplified the case in which errors in index values (bone mineral density, bone mineral content, and the like) include an error originating from scattered rays and an error originating from quantum noise or the like and the case in which errors in index values include an estimation accuracy error in the amount of scattered rays and an error originating from quantum noise or the like. However, the embodiments are not limited to these cases and may include a case in which one of an error originating from scattered rays, an error originating from quantum noise or the like, and an estimation accuracy error in the amount of scattered rays is set as an error in an index value (bone mineral density, bone mineral content, or the like).

The above embodiments have exemplified the case in which the X-ray diagnosis apparatus executes various types of processing. However, the embodiments are not limited to this, and a medical information processing apparatus may execute each processing described above.

FIG. 12 is a block diagram showing an example of the arrangement of a medical information processing apparatus 3 according to other embodiments. As shown in FIG. 12, the medical information processing apparatus 3 is connected to an X-ray diagnosis apparatus 1 via a network 2. The medical information processing apparatus 3 includes a communication interface 31, a memory 32, an input interface 33, a display 34, and a processing circuit 35. Note that the medical information processing apparatus 3 is an information processing apparatus such as a tablet terminal or a workstation.

The communication interface 31 is connected to the processing circuit 35 and controls the transmission and communication of various data with the X-ray diagnosis apparatus 1 or the like connected via a network. For example, the communication interface 31 is implemented by a network card, a network adapter, a network interface controller (NIC), or the like.

The memory 32 is connected to the processing circuit 35 and stores various data. For example, the memory 32 is implemented by a random access memory (RAM), a semiconductor memory device such as a flash memory, a hard disk, an optical disk, or the like. In this embodiment, the memory 32 stores, for example, the X-ray image data (the fluoroscopic image collected for positioning and the X-ray image collected by dual energy imaging) received from the X-ray diagnosis apparatus 1. The memory 32 also stores various types of information used for processing by the processing circuit 35, the processing result obtained by the processing circuit 35, and the like.

The input interface 33 is implemented by a trackball, switch buttons, a mouse, a keyboard, a touch pad that performs input operations based on touches on the operation screen, a touch monitor as a combination of a display screen and a touch pad, a non-contact input circuit using an optical sensor, a speech input circuit, and the like, which are used to, for example, perform various types of settings. The input interface 33 is connected to the processing circuit 35. The input interface 33 converts an input operation received from the operator and outputs the electrical signal to the processing circuit 35. Note that in this specification, the input interface 33 is not limited to the one including physical operation components such as a mouse and a keyboard. For example, the examples of the input interface include a processing circuit that receives an electrical signal corresponding to an input operation from an external input device provided separately from the apparatus and outputs the electrical signal to the control circuit.

The display 34 is connected to the processing circuit 35 and displays various types of information and various types of images output from the processing circuit 35. For example, the display 34 is implemented by a liquid crystal monitor, a cathode ray tube (CRT) monitor, a touch monitor, or the like. For example, the display 34 displays a user interface (UI) for receiving instructions from the operator, various images, and various processing results obtained by the processing circuit 35.

The processing circuit 35 controls each constituent element of the medical information processing apparatus 3 in accordance with an input operation received from the operator via the input interface 33. As shown in FIG. 12, the processing circuit 35 executes a control function 351, a calculation function 352, and a correction function 353. In this case, for example, the respective processing functions executed by the control function 351, the calculation function 352, and the correction function 353 which are constituent elements of the processing circuit 35 shown in FIG. 12 are recorded in the memory 32 in the form of programs that can be executed by a computer. The processing circuit 35 is, for example, a processor. The processing circuit 35 reads out programs from the memory 32 and executes the programs to implement functions corresponding to the programs. In other words, the processing circuit 35 that has read out each program has a corresponding function indicated in the processing circuit 35 in FIG. 12.

The control function 351 controls the overall medical information processing apparatus 3. The control function 351 also executes transmission/reception of data to/from the X-ray diagnosis apparatus 1 and processing similar to that executed by the control function 202 described above. The calculation function 352 executes processing similar to that executed by the calculation function 203 described above. The correction function 353 executes processing similar to that executed by the correction function 204 described above.

In the X-ray diagnosis apparatus described in each embodiment, each processing function is stored in the memory 17 in the form of a program that can be executed by a computer. The processing circuit 20 is a processor that reads out and executes each program from the memory 17 to implement a function corresponding to the program. In other words, the processing circuit 20 in a state in which each program is read out has a function corresponding to the read program. Although each embodiment has exemplified the case in which each processing function is implemented by the single processing circuit 20, the embodiments of the present invention are not limited to the above embodiments. For example, the processing circuit 20 may be formed by combining a plurality of independent processors and implement each processing function by making each processor execute a corresponding one of the programs. Alternatively, the respective processing functions of the processing circuit 20 may be implemented by being properly separated or integrated into a single or a plurality of processing circuits.

The term “processor” used in the above description refers to, for example, a circuit such as a central processing unit (CPU), a graphics processing unit (GPU), an application specific integrated circuit (ASIC), a programmable logic device (such as a simple programmable logic device (SPLD), a complex programmable logic device (CPLD), a field programmable gate array (FPGA)). The processor implements a function by reading out and executing a program stored in a storage 111.

Note that in each embodiment described above, the memory 17 stores the programs corresponding to the respective processing functions. However, a plurality of memories 17 may be separately arranged, and the processing circuit 20 may read out each program from a corresponding one of the memories 17. The programs may be directly incorporated in the circuit of the processor instead of being stored in the memory 17. In this case, the processor implements functions by reading out and executing programs incorporated in the circuit.

The constituent elements of the respective devices according to the above embodiments are functionally conceptual and need not necessarily be configured physically as shown in the accompanying drawings. That is, the specific form of separation/integration of the respective devices is not limited to that shown in the accompanying drawings, and can be functionally or physically separated or integrated partly or wholly according to various types of loads or usages. All or arbitrary some of the respective processing functions executed by the respective devices are implemented by a CPU or programs analytically executed by the CPU or implemented as wired-logic hardware.

Embodiment(s) of the present invention can also be realized by a computer of a system or apparatus that reads out and executes computer executable instructions (e.g., one or more programs) recorded on a storage medium (which may also be referred to more fully as anon-transitory computer-readable storage medium') to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)TM), a flash memory device, a memory card, and the like.

One of the embodiments described above enables proper evaluation of a time-series change in the state of the bone of an object.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2021-141010, filed Aug. 31, 2021, which is hereby incorporated by reference herein in its entirety.

What is claimed is:

1. An X-ray diagnosis apparatus comprising a display control unit configured to display information based on an error in an index value evaluating a state of a bone of an object based on at least one of a captured image of the object and an imaging condition which correspond to X-rays with two different types of energies.

2. The apparatus according to claim 1, further comprising a calculation unit configured to calculate an error in the index value,
wherein the calculation unit calculates an error in an index value evaluating a state of a bone of the object based on a geometric imaging condition in the imaging condition.

3. The apparatus according to claim 1, further comprising a calculation unit configured to calculate an error in the index value,
wherein the calculation unit calculates statistic information on the captured image as an error in an index value evaluating a state of a bone of the object.

4. The apparatus according to claim 2, wherein the calculation unit estimates an amount of scattered rays entering a region of interest based on the geometric imaging condition and calculates an error in the index value based on the estimated amount of scattered rays.

5. The apparatus according to claim 2, wherein the calculation unit estimates the error including an error based on quantum noise.

6. The apparatus according to claim 2, wherein the calculation unit estimates an amount of scattered rays inside a region of interest based on a position of the region of interest and calculates an error in the index value based on the estimated amount of scattered rays.

7. The apparatus according to claim 2, wherein the calculation unit calculates an error in the index value when a geometric imaging condition in the imaging condition is changed, and
the display control unit displays the error in the index value when the geometric imaging condition is changed.

8. The apparatus according to claim 4, further comprising a correction unit configured to correct an amount of scattered rays inside a region of interest based on a pixel value in a region other than an X-ray irradiation region determined by an X-ray aperture element,
wherein the calculation unit calculates an error in the index value based on the corrected amount of scattered rays.

9. The apparatus according to claim 1, further comprising a calculation unit configured to calculate an error in the index value,
wherein the calculation unit calculates an amount of scattered rays inside a region of interest based on a pixel value in a region other than an X-ray irradiation region determined by an X-ray aperture element.

10. The apparatus according to claim 1, further comprising a calculation unit configured to calculate an error in the index value,
wherein the calculation unit calculates an error in the index value based on an imaging condition for the captured image and executes determination on a condition used for capturing the captured image based on the calculated error and a past measured value corresponding to the error, and
the display control unit displays a determination result.

11. A medical information processing apparatus comprising a display control unit configured to display information based on an error in an index value evaluating a state of a bone of an object based on at least one of a captured image of the object and an imaging condition which correspond to X-rays with two different types of energies.

12. A medical information processing method comprising displaying information based on an error in an index value evaluating a state of a bone of an object based on at least one of a captured image of the object and an imaging condition which correspond to X-rays with two different types of energies.

13. A non transitory computer-readable storage medium storing a program for causing a computer to execute the method according to claim 12.
